(19) United States
(12) Patent Application Publication
(10) Pub. No.: US 2014/0336508 A1
(43) Pub. Date: Nov. 13, 2014

(54) IMAGE PROCESSING UNIT, ULTRASONIC IMAGING APPARATUS, AND IMAGE GENERATION METHOD FOR THE SAME

(71) Applicant: SAMSUNG ELECTRONICS CO., LTD., Suwon-si (KR)

(72) Inventors: Joo Young KANG, Yongin-si (KR); Sung Chan PARK, Suwon-si (KR); Kyu Hong KIM, Seongnam-si (KR); Jung Ho KIM, Yongin-si (KR); Su Hyun PARK, Hwaseong-si (KR)

(73) Assignee: SAMSUNG ELECTRONICS CO., LTD., Suwon-si (KR)

(21) Appl. No.: 14/271,778

(22) Filed: May 7, 2014

(30) Foreign Application Priority Data
May 7, 2013 (KR) 10-2013-0051205

(51) Int. Cl. G06T 5/20; A61B 8/08; A61B 8/00

(52) U.S. Cl. CPC G06T 5/20; A61B 8/461; A61B 8/5269
USPC 600/437; 382/131

(57) ABSTRACT

An image processing unit includes an input unit configured to receive image data of at least one image, a plurality of filters configured to filter the image data to generate a plurality of filtered images, and an image generator configured to compare the plurality of filtered images in a comparison to select at least one pixel from the plurality of filtered images according to results of the comparison.

### CROSS-REFERENCE TO RELATED APPLICATION(S)

This application claims priority from Korean Patent Application No. 2013-0051205, filed on May 7, 2013, in the Korean Intellectual Property Office, the disclosure of which is incorporated herein by reference in its entirety.

### BACKGROUND

1. Field

Apparatuses and methods consistent with exemplary embodiments relate to an image processing unit, an ultrasonic imaging apparatus, and an image generation method.

2. Description of the Related Art

Ultrasonic imaging apparatuses are imaging apparatuses that collect information of an object interior using ultrasonic waves and acquire an image of the interior of the object using the collected information.

In particular, an ultrasonic imaging apparatus may collect ultrasonic waves reflected or generated from a target site inside the object and acquire cross-sectional images of various tissues, structures or the like inside the object, e.g., cross-sectional images of various organs, soft tissues, or the like, using the collected ultrasonic waves. To implement such operation, the ultrasonic imaging apparatus may direct ultrasonic waves from an external source to the target site inside the object to collect ultrasonic waves reflected from the target site.

Ultrasonic imaging apparatuses may generate ultrasonic waves of a predetermined frequency using ultrasonic transducers or the like, direct the ultrasonic waves of a predetermined frequency to the target site, and receive ultrasonic waves reflected from the target site, thereby acquiring ultrasonic signals of a plurality of channels corresponding to the received ultrasonic waves. The ultrasonic imaging apparatuses may correct time differences between ultrasonic signals of a plurality of channels, focus the ultrasonic signals to obtain beamformed ultrasonic signals, and generate and acquire an ultrasonic image using the beamformed ultrasonic signals so that a user may obtain a cross-sectional image of the interior of the object.

The ultrasonic imaging apparatuses are smaller in size and less expensive than other imaging apparatuses, exhibit substantially real-time display of an image of the interior of the object, and have substantially no risk of exposure to radiation such as X-rays, and thus are widely used in a variety of fields, such as medical fields and the like.

### SUMMARY

One or more exemplary embodiments provide an image processing unit, an ultrasonic imaging apparatus, and an image generation method to improve image inaccuracy that may occur after applying a filter to an image in an image acquisition process.

One or more exemplary embodiments also provide an image processing unit, an ultrasonic imaging apparatus, and an image generation method that may attenuate a side lobe of a filtered image and maintain high-frequency components.

One or more exemplary embodiments further provide an image processing unit, an ultrasonic imaging apparatus, and an image generation method that may restore an image that substantially approximates to an ideal image.

Additional aspects will be set forth in part in the description which follows and, in part, will be obvious from the description, or may be learned by practice.

To address the above-described problems, there are provided an image processing unit, an ultrasonic imaging apparatus, and an image generation method.

In accordance with an aspect of an exemplary embodiment, an image processing unit includes an input unit configured to receive image data of at least one image, a plurality of filters configured to filter the image data to generate a plurality of filtered images, and an image generator configured to compare the plurality of filtered images in a comparison to select at least one pixel from the plurality of filtered images according to results of the comparison. In this regard, the image generator may include a selector configured to compare pixel data of a pixel of one of the plurality of filtered images with pixel data of corresponding pixels of remaining ones of the plurality of filtered images and select the at least one pixel from at least one of the plurality of filtered images. The selector may compare variances of pixel data of corresponding pixels of the plurality of filtered images and select a pixel having the smallest variance among the variances of the pixel data from the at least one of the plurality of filtered images.

In accordance with an aspect of another exemplary embodiment, an ultrasonic imaging apparatus includes a beamformer configured to beamform collected ultrasonic signals of a plurality of channels to output the beamformed ultrasonic signals, and an image processor configured to generate a plurality of filtered ultrasonic images by filtering at least one of the beamformed ultrasonic signals using a plurality of filters, to compare the plurality of filtered ultrasonic images, and to select at least one pixel from the plurality of filtered ultrasonic images according to results of the comparison.

In accordance with an aspect of still another exemplary embodiment, an image generation method includes receiving image data of at least one image, filtering the image data using a plurality of filters to generate a plurality of filtered images, and selecting at least one pixel from the plurality of filtered images according to results of comparison between the plurality of filtered images.

### BRIEF DESCRIPTION OF THE DRAWINGS

These and/or other aspects will become more apparent and readily appreciated from the following description of exemplary embodiments, taken in conjunction with the accompanying drawings in which:

FIG. 1 is a block diagram illustrating a configuration of an image processing unit according to an exemplary embodiment;

FIG. 2 is a block diagram illustrating a filtering unit according to an exemplary embodiment;

FIG. 3 illustrates example images obtained by various different filters;

FIG. 4 is a graph for explaining images acquired through filtering using various different filters;

FIG. 5 is a block diagram for explaining a point spread function;

FIG. 6 illustrates images for explaining a relationship among an original image, a radio frequency (RF) image, and deconvolution;

FIG. 7 is a block diagram illustrating a configuration of an image generator according to an exemplary embodiment;

FIG. 8 is a view for explaining an operation of an image generator according to an exemplary embodiment;

FIGS. 9 and 10 are graphs for explaining an image generated by an image generator according to an exemplary embodiment;

FIG. 11 is a flowchart illustrating an image processing method according to an exemplary embodiment;

FIG. 12 is a perspective view of an ultrasonic imaging apparatus according to an exemplary embodiment;

FIG. 13 is a block diagram illustrating a configuration of an ultrasonic imaging apparatus according to an exemplary embodiment;

FIG. 14 is a plan view of an ultrasonic probe according to an exemplary embodiment;

FIG. 15 is a view illustrating a configuration of a beamforming unit according to an exemplary embodiment;

FIG. 16 is a block diagram illustrating a configuration of an image processor of an ultrasonic imaging apparatus according to an exemplary embodiment; and

FIG. 17 is a flowchart illustrating a method of controlling an ultrasonic imaging apparatus according to an exemplary embodiment.

### DETAILED DESCRIPTION

Hereinafter, exemplary embodiments will now be described more fully with reference to the accompanying drawings. Like reference numerals refer to like elements throughout.

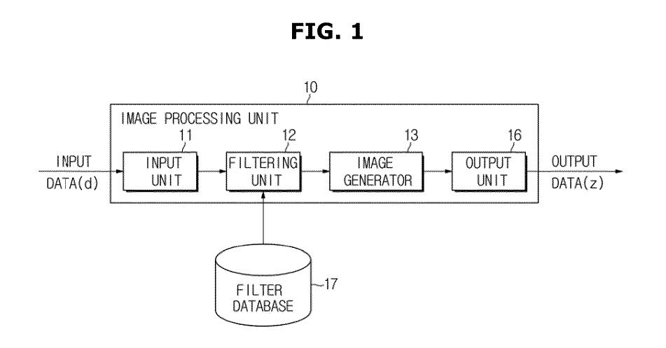

Hereinafter, an image processing unit according to an exemplary embodiment will be described with reference to FIGS. 1 to 10.

FIG. 1 is a block diagram illustrating a configuration of an image processing unit 10 according to an exemplary embodiment.

As illustrated in FIG. 1, the image processing unit 10 may receive predetermined input data d, generate predetermined output data z using the predetermined input data d, and output the generated predetermined output data z.

In particular, the image processing unit 10 may include an input unit 11, a filtering unit 12, an image generator 13, and an output unit 16, to perform processing of the received predetermined input data d.

Specifically, the input unit 11 receives the predetermined input data d transmitted from the outside.

According to one embodiment, the predetermined input data d input via the input unit 11 may be image data of at least one image. In particular, the input data d input via the input unit 11 may be image data acquired from waves with a predetermined frequency, such as sound waves, ultrasonic waves, or electromagnetic waves. For example, the input data d may be image data acquired from a predetermined electrical signal into which sound waves with an audible frequency (approximately 16 kHz or higher and approximately 20 kHz or less) are converted. In addition, the input data d may be image data acquired from a predetermined electrical signal into which sound waves with a frequency that is higher than the audible frequency, e.g., ultrasonic waves, are converted. In addition, the input data d may be image data acquired from a very high frequency (VHF) corresponding to a wavelength of about 1 m to about 10 m used in television (TV) or radio broadcasting and the like, or image data acquired using an ultra high frequency corresponding to a wavelength of about 10 cm to about 100 cm or the like used in a radar and the like. In addition, the input data d may be image data acquired using various other methods.

The input unit 11 transmits the input data d to the filtering unit 12.

The filtering unit 12 filters the input data d using a predetermined filter to acquire a predetermined filtered signal. In one embodiment, the filtering unit 12 may filter the input data d using a plurality of filters. Accordingly, the filtering unit 12 may acquire a plurality of filtered signals from the predetermined input data d.

In addition, as illustrated in FIG. 1, the filtering unit 12 may read a filter database 17, detect a predetermined filter from the filter database 17, and filter the input data d using the detected predetermined filter.

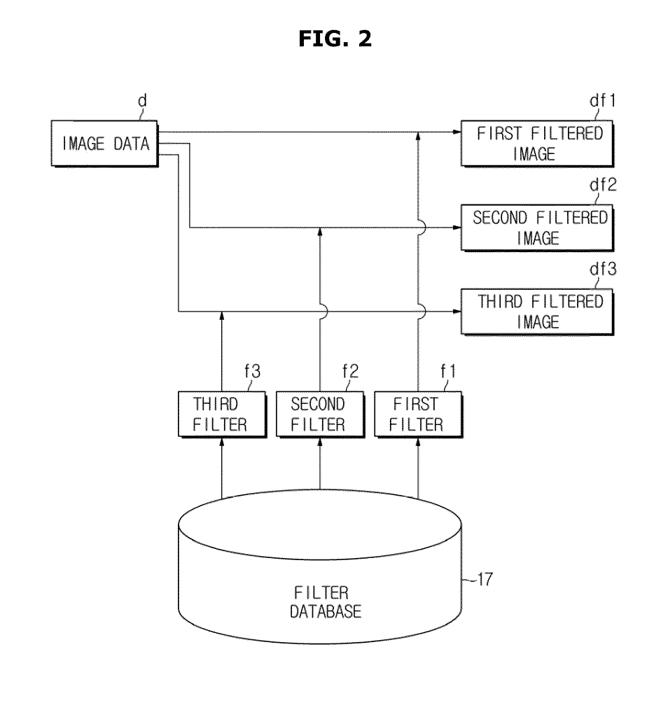

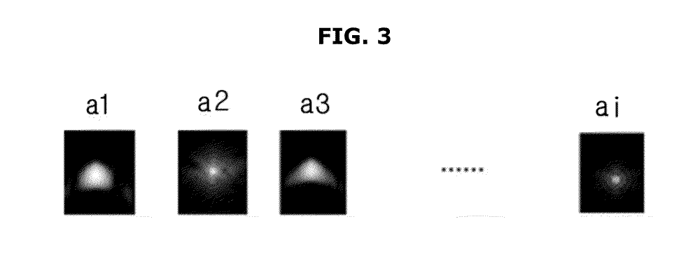

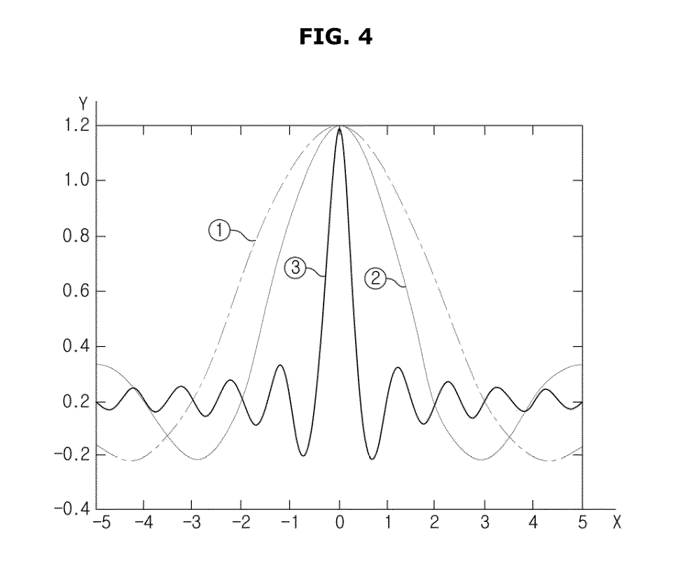

FIG. 2 is a block diagram illustrating the filtering unit 12 according to an exemplary embodiment. FIG. 3 illustrates example images obtained by various different filters. FIG. 4 is a graph for explaining images acquired through filtering using various different filters.

As illustrated in FIG. 2, the filtering unit 12 may acquire a plurality of filtered images df1, df2 and df3 using a plurality of filters, e.g., first, second and third filters f1, f2 and f3. More specifically, the filtering unit 12 may acquire image data of a plurality of filtered images, e.g., image data of first, second and third filtered images df1, df2 and df3 by applying predetermined filters, e.g., the first, second and third filters f1, f2 and f3 to the input data d, e.g., image data d of a predetermined image. That is, the filtering unit 12 may acquire a plurality of filtered images df1, df2 and df3 by acquiring the first filtered image df1 by applying the first filter f1 to an image corresponding to the received image data d, acquiring the second filtered image df2 using the second filter f2 to the image corresponding to the received image data d, and acquiring the third filtered image df3 using the third filter f3 to the image corresponding to the received image data d. Although FIG. 2 illustrates that the filtering unit 12 acquires the three kinds of filtered images, i.e., the first, second and third filtered images df1, df2 and df3 from a single image using the first, second and third filters f1, f2 and f3, the number of filters or the number of filtered images acquired using the filters may be varied.

The plurality of filters used in the filtering unit 12, e.g., the first, second and third filters f1, f2 and f3, may be different from one another. That is, the filters of the filtering unit 12 may be different filters to filter the same image to acquire different images, e.g., images a1, a2, a3, . . . , ai shown in FIG. 3. Accordingly, the first, second and third filtered images df1, df2 and df3 acquired using the first, second and third filters f1, f2, f3, which are different from one another, may be different from one another as illustrated in FIG. 4.

In addition, when the first, second and third filters f1, f2 and f3 of the filtering unit 12 are applied to the received input data d, a signal of a main lobe of the image data d may be emphasized according to the filter used. Types of the main lobe of the image data d may vary according to kinds of filter. In addition, while the filtering unit 12 performs a filtering process, a side lobe in the vicinity of the main lobe may also be emphasized by various external factors, e.g., the velocity of sound waves, a distance between an object detected by sound waves or the like and the image processing unit or the like.

FIG. 4 is a graph showing different overlapping filtered image data acquired when the same image data are filtered using three different filters. In FIG. 4, first, second and third curves \textcircled{1} to \textcircled{3} respectively show filtered image data acquired by applying different filters, e.g., the first, second and third filters f1, f2 and f3 to the same image data. Referring to FIG. 4, a central portion of each curve denotes a main lobe, and a peripheral portion of each curve denotes a side lobe.

As illustrated in FIG. 4, the first curve \textcircled{1} for the first filtered image data df1 has an upwardly protruding central portion while extended on left and right sides relative to the central portion. That is, the first filtered image data df1 acquired by the first filter f1 has higher values both in the central portion and the peripheral portion. Thus, the first filtered image data df1 acquired by filtering the input data d using the first filter f1 may be image data with an emphasized main lobe and an emphasized side lobe.

The second curve \textcircled{2} for the second filtered image data df2 has an upwardly protruding central portion in a sharper shape than that of the first curve \textcircled{1} as illustrated in FIG. 4. In addition, the second curve \textcircled{2} has a downwardly protruding shape at middle portions of the respective right and left sides of the central portion and thus it can be seen that the second curve \textcircled{2} has higher values in end portions of the respective right and left sides. Thus, the second filtered image data df2 is emphasized in the central portion and the end portions of a peripheral region. In other words, the second filtered image data df2 may be image data with both an emphasized main lobe and emphasized end portions of the side lobe.

The third curve \textcircled{3} for the third filtered image data df3 has a central portion having a sharp, upwardly protruding shape as illustrated in FIG. 4. A peripheral region of the third curve \textcircled{3} has a repetitive wave shape. That is, the third filtered image data df3 may be image data, a main lobe of which is emphasized and a side lobe of which is partially emphasized.

As described above, the filtering unit 12 may acquire a plurality of filtered images, e.g., the first, second and third filtered images df1, df2 and df3, using a plurality of filters, e.g., the first, second and third filters f1, f2 and f3. In this case, a plurality of different filtered images, e.g., the first, second and third filtered images df1, df2 and df3, may be acquired using a plurality of different filters, e.g., the first, second and third filters f1, f2 and f3.

In one embodiment, the filters used in the filtering unit 12, e.g., the first, second and third filters f1, f2 and f3, may be higher resolution filters, least square filters (LSFs), or cepstrum filters.

In one embodiment, at least one of the filters used in the filtering unit 12 may be a point spread function (PSF).

The PSF is a function related to a relationship between an ideal image and an acquired image signal (e.g., radio frequency (RF) image data) and used for restoration of an ideal image by correcting errors due to technical characteristics, physical characteristics or the like of an imaging apparatus or the like.

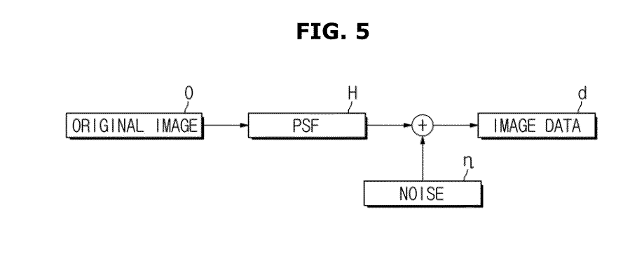

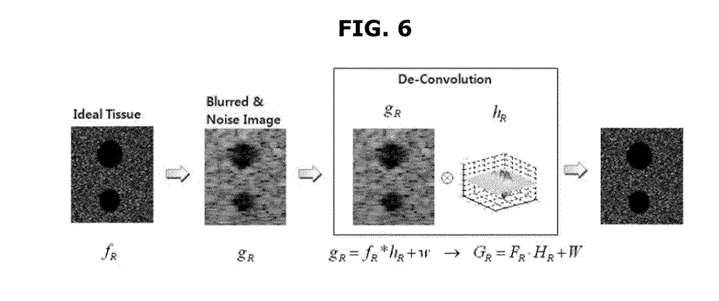

FIG. 5 is a block diagram for explaining a PSF. FIG. 6 illustrates images for explaining a relationship among an original image, image data in an ultrasonic imaging apparatus, and deconvolution according to an exemplary embodiment.

When an imaging apparatus acquires an image of an object, image data d that are different from an original image O may be output due to technical characteristics, physical characteristics, or noise η of the imaging apparatus. In other words, the image data d acquired by the imaging apparatus may be a signal output after the original image O is changed according to the technical characteristics or physical characteristics of the imaging apparatus and the noise q applied thereto.

The PSF will now be described in more detail with reference to FIG. 6. FIG. 6 illustrates image data acquired by an ultrasonic imaging apparatus. An image f_{R} of FIG. 6 shows an ideal shape of a tissue in a human body. When the ideal image is given as f_{R} illustrated in FIG. 6, a beamformed ultrasonic image acquired by the ultrasonic imaging apparatus may be an image g_{R} of FIG. 6. That is, the original image f_{R} becomes different from the acquired image g_{R} according to a sound speed of ultrasonic waves, an inner depth of a target site of an object from which ultrasonic waves are reflected, or the like.

Thus, when the original image O is restored using the image data d, a difference between the original image O and the image data d needs to be corrected to acquire an accurate image of a target site to be imaged. In this case, on the premise that a predetermined relationship exists between the original image O and the acquired image data d, image restoration is performed by correcting the image data d using an inverse function to a predetermined function corresponding to the predetermined relationship. In this regard, the predetermined function is a point spread function H.

A relationship among the original image O, the point spread function H, the noise η, and the image data d, as illustrated in FIG. 5, may be represented by Equation 1 below:

d=H·o+η [Equation 1]

wherein d denotes output image data, H denotes a point spread function, x denotes a signal for the original image O, and η denotes noise.

In a case where no noise exists, the image data d may be represented by a product of the original image O and the point spread function H. Thus, when an appropriate point spread function H for measured image data d is identified, the original image O may be acquired from the image data d. That is, when the point spread function H for the image data d is identified, an image that is the same as or substantially similar to the original image O of an object may be restored.

The filtering unit 12 may restore and acquire at least one filtered image using the above-described point spread function. In particular, the filtering unit 12 may generate a filtered image that is the same or substantially the same as the original image O using the image data d and the appropriate point spread function H. For example, as illustrated in FIG. 6, the filtering unit 12 may acquire a restored image (the rightmost image of FIG. 6) that is the same as or substantially similar to the original image O (f_{R}) through deconvolution by applying an inverse function to an appropriate point spread function H_{R} to the image data d (g_{R}).

According to one embodiment, as illustrated in FIG. 2, the filtering unit 12 may acquire the first, second and third filtered image data df1, df2 and df3 by calling a plurality of filters from the filter database 17, e.g., the first, second and third filters f1, f2 and f3, the point spread function, or the like and applying the called filters, e.g., the first, second and third filters f1, f2 and f3, the called point spread function, or the like to the predetermined image data d.

The filter database 17 may comprise various types of filters as illustrated in FIG. 4 or a point spread function.

The filtering unit 12 may acquire the first, second and third filtered image data df1, df2 and df3 by selecting predetermined filters, e.g., the first, second and third filters f1, f2 and f3 from among the various types of filters stored in the filter database 17, calling the selected predetermined filters, and applying the called filters to the image data d. The filtering unit 12 may select the predetermined filters according to predefined settings, or instructions or commands input by a user. In addition, the filtering unit 12 may select the predetermined filters according to the input image data d or regardless of the input image data d.

The first, second and third filtered image data df1, df2 and df3 acquired by the filtering unit 12 may be transmitted to the image generator 13.

The image generator 13 extracts pixel data for each of a plurality of pixels from the first, second and third filtered image data df1, df2 and df3 to generate a new image.

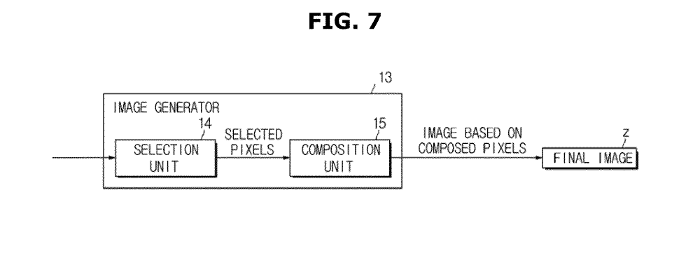

FIG. 7 is a block diagram illustrating a configuration of the image generator 13, according to an exemplary embodiment.

As illustrated in FIG. 7, the image generator 13 may include a selection unit 14 to select at least one pixel and a composition unit 15 to generate a final image z based on the at least one pixel.

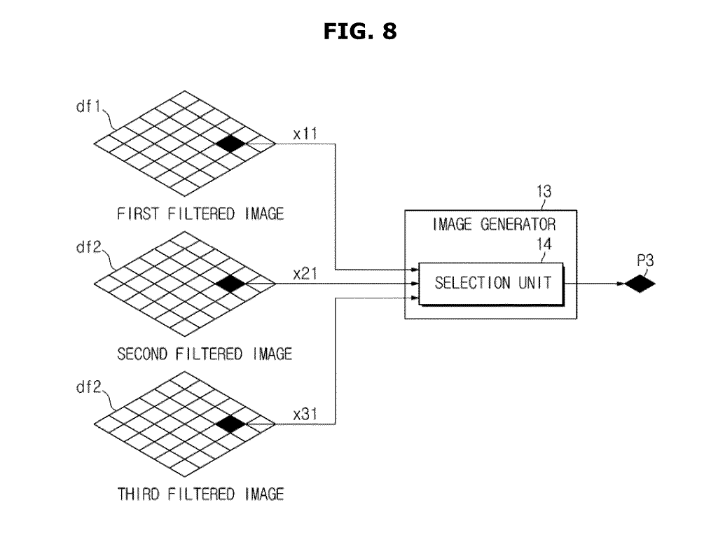

FIG. 8 is a view for explaining an operation of an image generator 13 according to an exemplary embodiment.

As illustrated in FIG. 2, when a plurality of filtered images, e.g., the first, second and third filtered image data df1, df2 and df3 are acquired, the selection unit 14 of the image generator 13 may select at least one pixel from the filtered images, e.g., the first, second and third filtered image data df1, df2 and df3 and extract pixel data therefrom.

Each of the filtered images, e.g., the first, second and third filtered images df1, df2 and df3 may comprise a plurality of pixels, i.e., dots, which are minimum units for image display. The selection unit 14 may compare the filtered images with each other and select at least one pixel from the filtered images according to comparison results.

In one embodiment, as illustrated in FIG. 8, the selection unit 14 may compare data of corresponding pixels, e.g., first, second and third pixels x11, x21 and x31 of the respective filtered images, e.g., the first, second and third filtered image data df1, df2 and df3 and select at least one pixel, e.g., the third pixel x31 from at least one (e.g., the third filtered image df3) of the filtered images, e.g., the first, second and third filtered images df1, df2 and df3 according to comparison results. That is, the selection unit 14 may select at least one (e.g., the third pixel x31) of the pixels of the filtered images, e.g., the first, second and third pixels x11, x21 and x31 of the respective first, second and third filtered images df1, df2 and df3 according to comparison results. Pixel data P3 of the selected pixel of the filtered images, e.g., the third pixel x31, is provided to be used for the final image generated by the image generator 13.

As described above, the corresponding pixels of the respective filtered images may be compared with one another. The corresponding pixels of the respective filtered images may be pixels corresponding to the same pixel of the image data d prior to the filtering process. The pixels selected by the selection unit 14 may be used to constitute the image to be generated by the composition unit 15.

In one embodiment, the selection unit 14 may compare variances of pixels of the filtered image data, e.g., the first, second and third filtered image data df1, df2 and df3, and select a pixel of at least one of the filtered images according to comparison results.

In this case, according to one embodiment, the selection unit 14 may calculate variances of corresponding pixel data of the filtered images and select at least one pixel having the smallest variance among the calculated variances of the pixel data from the corresponding pixels of the filtered images.

In addition, in another embodiment, the selection unit 14 may calculate variances of pixel data of corresponding pixels and adjacent pixels of the filtered images, and select a pixel having the smallest variance among the calculated variances of the corresponding pixels of the filtered images or select a pixel having the smallest variance among the calculated variances of the corresponding pixels and adjacent pixels of the filtered images.

The selection unit 14 may select at least one pixel of the filtered images from among the corresponding pixels of the plurality of filtered images using Equation 2 below:

$$\hat{x} = {arg min}_{{f}_{( i )}} {‖ {f}_{( i )}^{- 1} \cdot y - x ‖}^{2} \qquad [ Equation 2 ]$$

wherein x denotes an ideal ultrasonic image, and y denotes image data d, and f₍ᵢ₎ denotes an i-th filter or a point spread function.

According to Equation 2, when deconvolution is performed by applying an inverse of the filter or the point spread function, i.e., an inverse filter or an inverse point spread function to the image data d, an appropriate (e.g., optimum) filter or point spread function for minimizing the square of a difference between deconvolution results and the ideal image may be detected. That is, the optimum filter or point spread function may be detected by minimum variance. Consequently, an appropriate filter may be selected from among a plurality of filters, e.g., the first, second and third filters f1, f2 and f3.

As described above, the filtering unit 12 may acquire a plurality of filtered images, e.g., the first, second and third filtered images df1, df2 and df3 using a plurality of filters, e.g., the first, second and third filters f1, f2 and f3. The selection unit 14 may determine an appropriate (e.g., optimum) filtered image of the filtered images acquired by the filtering unit 12, e.g., the third filtered image df3, using Equation 2 or 3 above, and detect predetermined pixels from the determined optimum filtered image, e.g., the third filtered image df3 to select predetermined pixels for an entire area or a partial area of an image to be generated by the composition unit 15.

In one embodiment, the selection unit 14 may determine a filtered image to which an optimal filter has been applied, for a portion of the image to be generated by the composition unit 15. In this case, the selection unit 14 may detect predetermined pixels to be arranged in a particular location of a predetermined image to be composed by the composition unit 15 from the determined filtered image to which an optimal filter has been applied so that optimal pixels for constituting a portion of the image to be composed by the composition unit 15 are selected and detected from the plurality of filtered images.

In a case where the ideal ultrasonic image is unidentified and thus represented as 0, Equation 2 may be represented by Equation 3 below:

$$\hat{x} = {arg min}_{{f}_{( i )}} {‖ {f}_{( i )}^{- 1} \cdot y ‖}^{2} \qquad [ Equation 3 ]$$

According to Equation 3, calculation of the optimal filter or point spread function corresponds to selection of a filter or a point spread function by which a minimum value of a deconvolution result of the filter or the point spread function and the image data d is obtained. The selection unit 14 may select at least one pixel of the filtered images from among corresponding pixels of the plurality of filtered images using Equation 3 above.

Consequently, the selection unit 14 detects a plurality of pixels for constituting the image to be composed by the composition unit 15 from the plurality of filtered images, e.g., the first, second and third filtered images df1, df2 and df3.

Pixel data of each pixel selected by the selection unit 14 are transmitted to the composition unit 15, and the composition unit 15 composes pixel data of each pixel to generate at least one image.

The at least one image generated by the composition unit 15 may be acquired by composing portions of images filtered using different filters, e.g., portions of the first, second and third filtered images df1, df2 and df3. For example, pixel data of some of the pixels of the at least one image generated by the composition unit 15 may be data of pixels of a predetermined filtered image, e.g., the first filtered image df1, and pixel data of others thereof may be data of pixels of a filtered image that is different from the predetermined filtered image, e.g., the second filtered image df2. In some embodiments, all pixels of the image generated by the composition unit 15 may be selected from among pixels of an image filtered using a particular filter.

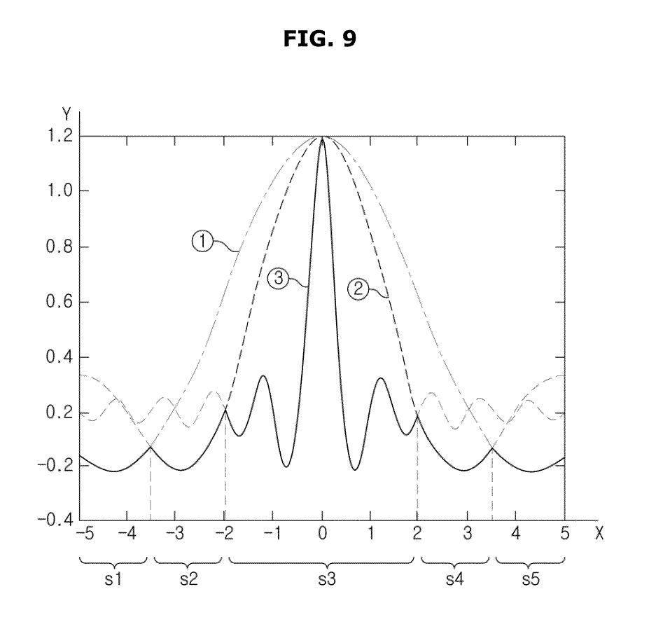

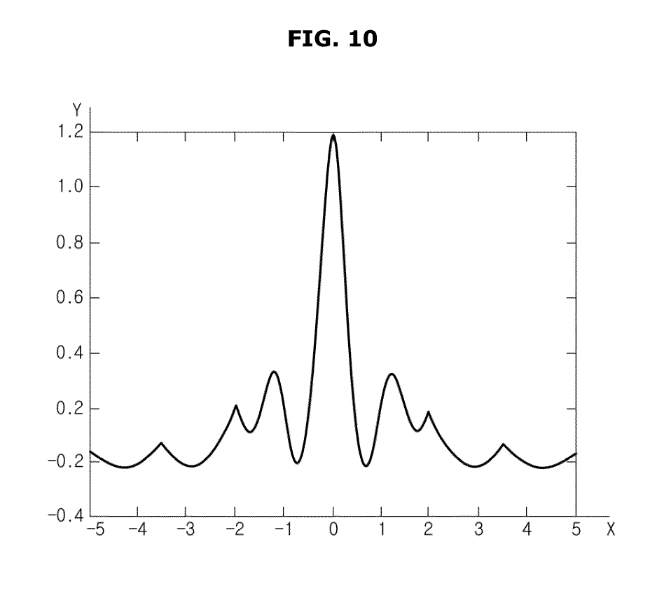

FIGS. 9 and 10 are graphs for explaining an image generated by the image generator 13 according to an exemplary embodiment.

FIG. 9 illustrates the first, second and third filtered images df1, df2 and df3 obtained using three kinds of filters, e.g., the first, second and third filters f1, f2 and f3 as illustrated in FIG. 4. As illustrated in FIG. 9, the first curve \textcircled{1} for the first filtered image df1 has smaller values in s1 and s5 sections than those of other curves, i.e., the second and third curves \textcircled{2} and \textcircled{3}. In s2 and s4 sections, the second curve \textcircled{2} for the second filtered image df2 has smaller values than those of the other curves. The third curve \textcircled{3} for the third filtered image df3 has smaller values in an s3 section than those of the other curves. In this case, the selection unit 14 may select appropriate (e.g., optimum) pixels using the above-described method and the composition unit 15 may compose the selected pixels, thereby acquiring output image data z having a shape as illustrated in FIG. 10. That is, an optimal image with improved quality may be acquired.

With reference to FIG. 10, in the acquired optimal image, a shape of a main lobe is maintained or emphasized and a side lobe is attenuated to, e.g., a minimum level. Consequently, the side lobe of the filtered image may be attenuated and high-frequency components thereof may be maintained. In addition, when an image is acquired using sound waves, a VHF, or the like, blurring or the like according to the velocity of sound waves, VHF, or the like may be substantially prevented. Therefore, an image of an object with improved accuracy may be acquired.

The at least one image data z composed by the composition unit 15 are transmitted to the output unit 16, and the output unit 16 may transmit the image data z to an external device, e.g., a display device or the like.

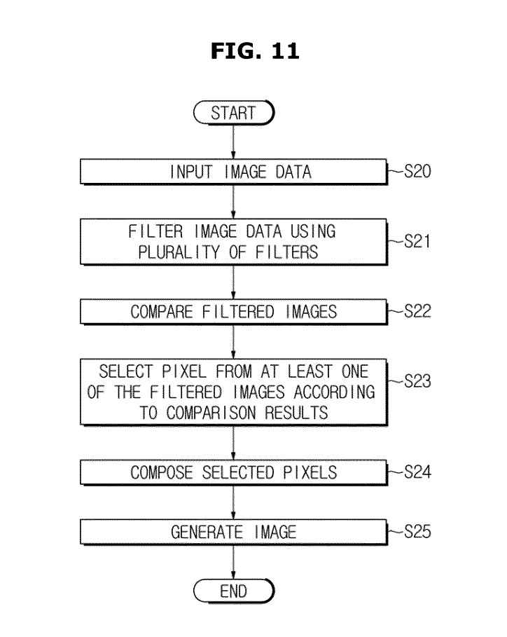

Hereinafter, an image processing method according to an exemplary embodiment will be described with reference to FIG. 11.

FIG. 11 is a flowchart illustrating an image processing method according to an exemplary embodiment.

According to the image processing method illustrated in FIG. 11, first, image data are input (operation S20). The input image data are filtered using a plurality of different filters to acquire a plurality of different filtered images (operation S21). In one embodiment, at least one of the filters may be a point spread function. In addition, in some embodiments, a process of calling the different filters from a filter database may be performed before operation S20. In addition, the filtered images are compared with each other (operation S22), and at least one pixel is selected from among at least one of the filtered images according to comparison results (operation S23). In one embodiment, pixel data of at least one corresponding pixel of each filtered image may be compared and at least one pixel may be selected from at least one of the filtered image. In this case, a pixel may be selected, which has the smallest variance among the variances of the pixel data of at least one corresponding pixel of each filtered image. In this regard, Equations 2 and 3 above may be used.

When at least one pixel is selected from the filtered data, the selected pixel is composed (operation S24) to generate at least one image (operation S25). As a result, as illustrated in FIG. 10, an image, a main lobe of which is emphasized and a side lobe of which is attenuated, may be acquired.

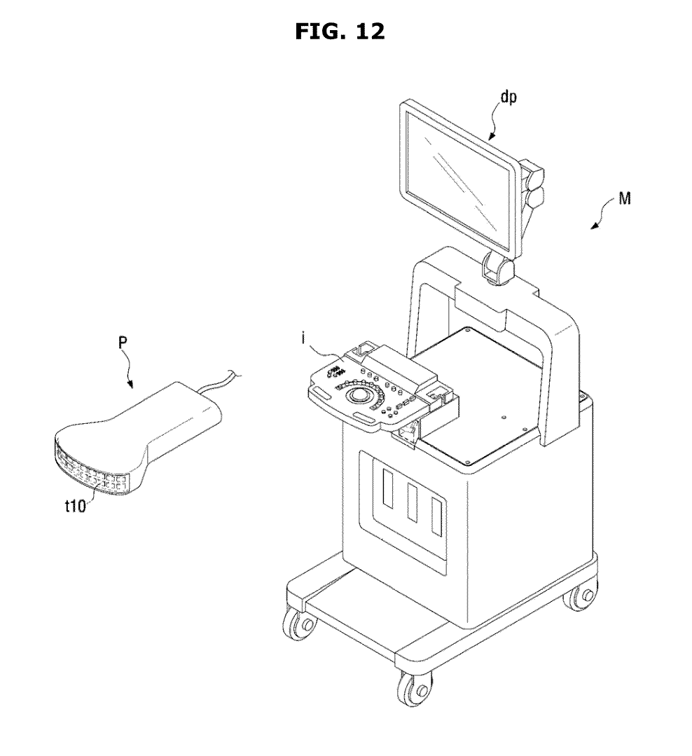

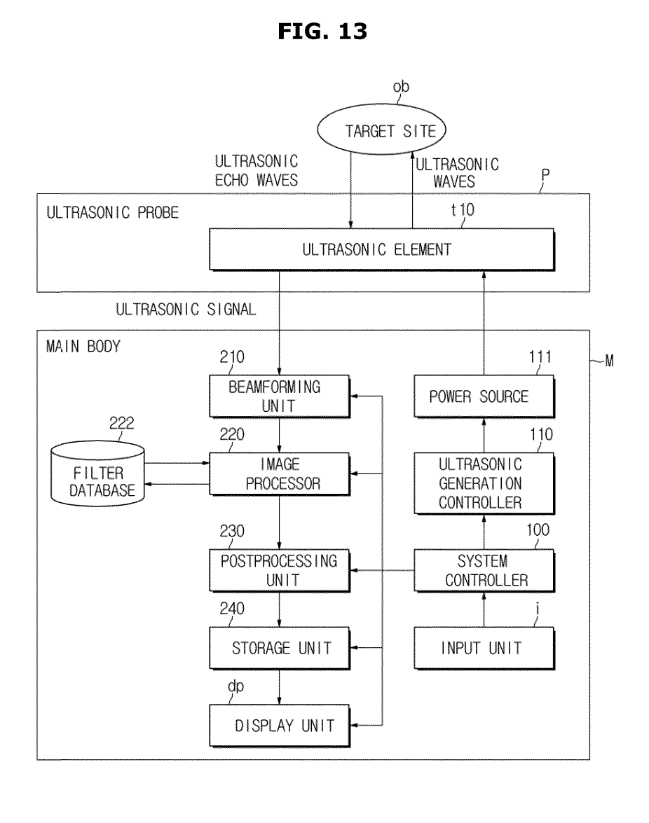

Hereinafter, an ultrasonic imaging apparatus according to an exemplary embodiment will be described with reference to FIGS. 12 to 17. FIG. 12 is a perspective view of an ultrasonic imaging apparatus according to an exemplary embodiment. FIG. 13 is a block diagram illustrating a configuration of an ultrasonic imaging apparatus according to an exemplary embodiment.

As illustrated in FIGS. 12 and 13, according to one embodiment, the ultrasonic imaging apparatus may include an ultrasonic probe P to collect an ultrasonic signal of the inside of an object ob using ultrasonic waves, and a main body M to generate an ultrasonic image using the ultrasonic signal collected by the ultrasonic probe P.

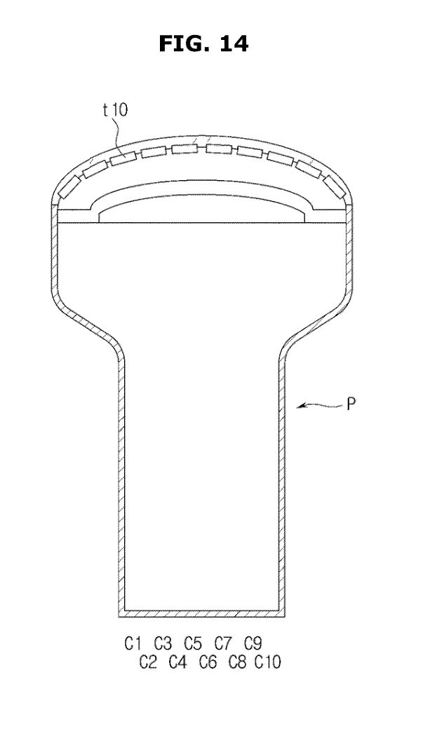

FIG. 14 is a plan view of the ultrasonic probe P according to an exemplary embodiment.

As illustrated in FIGS. 12 to 14, the ultrasonic probe P may include at least one ultrasonic element t10 to generate ultrasonic waves according to applied power, direct the generated ultrasonic waves to at least one target site inside the object ob, receive ultrasonic echo waves reflected from the at least one target site of the object ob, and convert the ultrasonic echo waves into an electrical signal. The at least one ultrasonic element t10 may be installed at an end portion of the ultrasonic probe P as illustrated in FIG. 14. In this case, the at least one ultrasonic element t10 may be disposed at an end portion of the ultrasonic probe P in at least one row.

In some embodiments, the at least one ultrasonic element t10 may be any one of at least one ultrasonic generation element (not shown) to generate ultrasonic waves according to applied power and at least one ultrasonic receiving element (not shown) to receive ultrasonic echo waves and convert the received ultrasonic echo waves into an electrical signal. In other embodiments, the ultrasonic element t10 may generate ultrasonic waves and receive ultrasonic echo waves.

The ultrasonic element t10 or the ultrasonic generation element may vibrate according to a pulse signal or alternating current applied thereto under control of an ultrasonic generation controller 110 installed at the ultrasonic probe P or the main body M to generate ultrasonic waves. The generated ultrasonic waves are directed to the target site inside the object ob. In some embodiments, the ultrasonic waves generated by the ultrasonic element t10 may be directed by focusing on a plurality of target sites inside of the object. That is, the generated ultrasonic waves may be directed by multi-focusing.

The ultrasonic waves generated by the ultrasonic element t10 are reflected from the at least one target site inside the object ob to return to the ultrasonic element t10. The ultrasonic element t10 or the ultrasonic receiving element receives ultrasonic echo waves reflected from the at least one target site. When the ultrasonic echo waves reach the ultrasonic element t10 or the ultrasonic receiving element, the ultrasonic element t10 or the ultrasonic receiving element vibrates with a predetermined frequency corresponding to a frequency of the ultrasonic echo waves to output alternating current of a frequency corresponding to the vibration frequency of the ultrasonic element t10 or the ultrasonic receiving element. Accordingly, the ultrasonic element t10 or the ultrasonic receiving element may convert the received ultrasonic echo waves into a predetermined electrical signal.

Since each ultrasonic element t10 or each ultrasonic receiving element receives external ultrasonic waves and outputs an electrical signal into which the ultrasonic waves have been converted, the ultrasonic probe P may output electrical signals of a plurality of channels C1 to C10 as illustrated in FIG. 14. In this case, the number of channels may be, for example, 64 to 128.

The ultrasonic element t10 may be an ultrasonic transducer. A transducer is a device to convert a predetermined type of energy into another type of energy. For example, an ultrasonic transducer may convert electrical energy into wave energy or vice versa. Accordingly, the ultrasonic transducer may function as a combination of the ultrasonic element t10, the ultrasonic generation element, and the ultrasonic receiving element.

More particularly, the ultrasonic transducer may include a piezoelectric vibrator or a thin film. When alternating current is applied to piezoelectric vibrators or thin films of the ultrasonic transducers from a power source 111 such as an external power supplier or an internal electrical storage device, e.g., a battery or the like, the piezoelectric vibrators or thin films vibrate with a predetermined frequency according to applied alternating current and ultrasonic waves of the predetermined frequency are generated according to the vibration frequency. On the other hand, when ultrasonic echo waves of the predetermined frequency reach the piezoelectric vibrators or thin films, the piezoelectric vibrators or thin films vibrate according to the ultrasonic echo waves. In this regard, the piezoelectric vibrators or thin films output alternating current of a frequency corresponding to the vibration frequency thereof.

The ultrasonic transducer may be, for example, any one of a magnetostrictive ultrasonic transducer using a magnetostrictive effect of a magnetic body, a piezoelectric ultrasonic transducer using a piezoelectric effect of a piezoelectric material, and a capacitive micromachined ultrasonic transducer (cMUT), which transmits and receives ultrasonic waves using vibration of several hundreds or several thousands of micromachined thin films. In addition, other kinds of transducers that generate ultrasonic waves according to an electrical signal or generate an electrical signal according to ultrasonic waves may also be used as the ultrasonic transducer.

As illustrated in FIG. 13, the main body M may include a system controller 100, the ultrasonic generation controller 110, the power source 111, a beamforming unit 210, an image processor 220, a filter database 222, an image postprocessing unit 230, a storage unit 240, an input unit i, and a display unit dp.

The system controller 100 controls overall operations of the main body M. In particular, the system controller 100 may generate a predetermined control signal for each element of the main body M as illustrated in FIG. 13, e.g., the ultrasonic generation controller 110, the beamforming unit 210, the image processor 220, the image postprocessing unit 230, the storage unit 240, the display unit d, and the like, thereby controlling an operation of each element of the main body M. The system controller 100 may include a processor, a microprocessor, a central processing unit (CPU), or an integrated circuit for executing programmable instructions. The storage unit 240 may include a memory.

In some embodiments, the system controller 100 may control the ultrasonic imaging apparatus by generating a predetermined control command for each element of the main body M according to predetermined settings or separate instructions or commands input by the user via the input unit i.

The ultrasonic generation controller 110 may receive predetermined control commands from the system controller 100 or the like, generate predetermined control signals according to the received control commands, and transmit the control signals to the ultrasonic elements t10 of the ultrasonic probe P. In this case, the ultrasonic elements t10 may generate ultrasonic waves by operating according to the transmitted predetermined control signals. In addition, the ultrasonic generation controller 110 may generate a control signal for the power source 111 electrically connected to the ultrasonic element t10 according to the received control command and transmit the generated control signal to the power source 111. In this case, the power source 111 having received the control signal may supply alternating current of a predetermined frequency to the ultrasonic elements t10 according to the control signal so that the ultrasonic elements t10 generate ultrasonic waves of a frequency corresponding to the frequency of the alternating current.

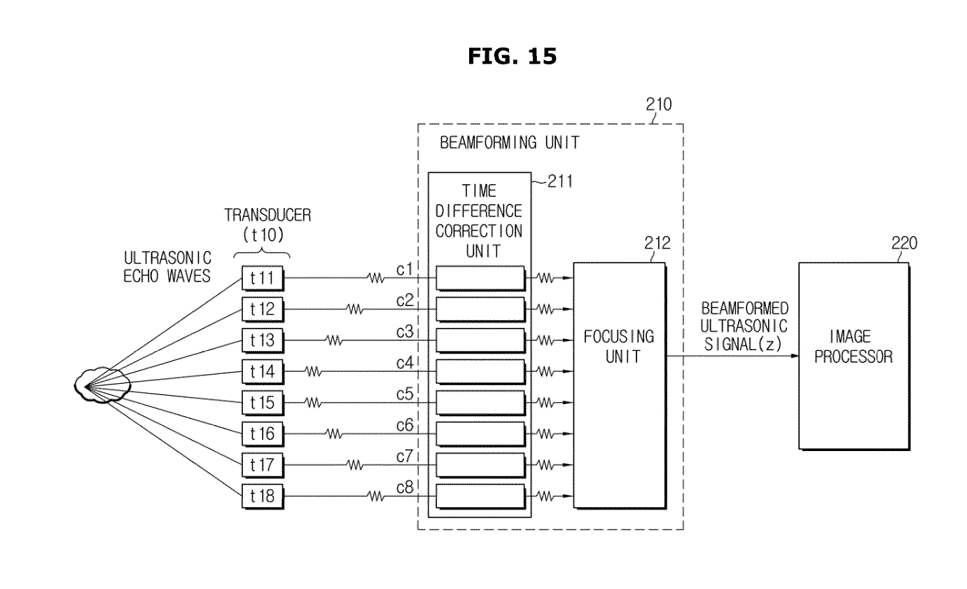

FIG. 15 is a view illustrating the beamforming unit 210 according to an exemplary embodiment.

The beamforming unit 210 of the main body M receives ultrasonic signals of a plurality of channels, e.g., channels c1 to c8, from the ultrasonic elements t10, focuses the received ultrasonic signals of the channels c1 to c8, and outputs the beamformed ultrasonic signals. The beamformed ultrasonic signals may constitute an ultrasonic image. In particular, the beamforming unit 210 may perform beamforming to estimate the size of reflected waves in a specific space for the ultrasonic signals of the channels C1 to C8.

In one embodiment, as illustrated in FIG. 15, the beamforming unit 210 may include a time difference correction unit 211 and a focusing unit 212.

The time difference correction unit 211 may correct time differences among ultrasonic signals output from respective ultrasonic elements t11 to t18 of the ultrasonic element t10.

As described above, the ultrasonic elements t10 receive ultrasonic echo waves reflected from a target site. While distances between the target site and each of the ultrasonic elements t11 to t18 installed at the ultrasonic probe P are different, the sound velocities of ultrasonic waves are substantially constant in the same media. Thus, the ultrasonic elements t11 to t18 receive ultrasonic echo waves generated or reflected from the same target site at different times. Accordingly, although the ultrasonic elements t11 to t18 receive the same ultrasonic echo waves, predetermined time differences occur between ultrasonic signals output from the ultrasonic elements t11 to t18. The time difference correction unit 211 may correct the time differences between the ultrasonic signals output from the ultrasonic elements t11 to t18.

To correct the time differences between the ultrasonic signals, as illustrated in FIG. 15, the time difference correction unit 211 may delay transmission of ultrasonic signals to be input to particular channels, e.g., the channels c1 to c8, to some extent according to predetermined settings so that the ultrasonic signals of the channels c1 to c8 are transmitted to the focusing unit 212 substantially at the same time.

The focusing unit 212 may focus ultrasonic signals. As illustrated in FIG. 15, the focusing unit 212 may focus the ultrasonic signals of the channels c1 to c8 in which time differences therebetween have been corrected.

The focusing unit 212 may focus ultrasonic waves by adding a predetermined weight, e.g., a beamforming coefficient, to each input ultrasonic wave to emphasize or relatively attenuating an ultrasonic signal at a predetermined location. Accordingly, an ultrasonic image according to user needs may be generated.

In one embodiment, the focusing unit 212 may focus ultrasonic signals using a predefined beamforming coefficient regardless of the ultrasonic signals. In another embodiment, the focusing unit 212 may obtain an appropriate beamforming coefficient based on the input ultrasonic signals and focus the ultrasonic signals using the obtained beamforming coefficient.

A beamforming process performed in the time difference correction unit 211 and the focusing unit 212 may be represented by Equation 4 below:

$$z [ n ] = {\sum }_{m = 0}^{M - 1} {w}_{m} [ n ] {x}_{m} [ n - {Δ}_{m} [ n ] ] \qquad [ Equation 4 ]$$

wherein n is an index for a location, e.g., a depth of the target site, m is an index for each channel to which an ultrasonic signal is input, and wₘ denotes a weight to be applied to an ultrasonic signal of an m-th channel, e.g., a beamforming coefficient wₘ for the m-th channel. In addition, Δₘ denotes time difference correction values. The time difference correction values may be used to delay transmission time of ultrasonic signals, which is performed by the time difference correction unit 211. According to Equation 4 above, the focusing unit 212 focuses the ultrasonic signal of each channel in which time differences therebetween have been corrected to output a beamformed ultrasonic signal, e.g., a beamformed ultrasonic image z.

The ultrasonic signals z beamformed by the beamforming unit 210 are transmitted to the image processor 220 as illustrated in FIGS. 14 and 15.

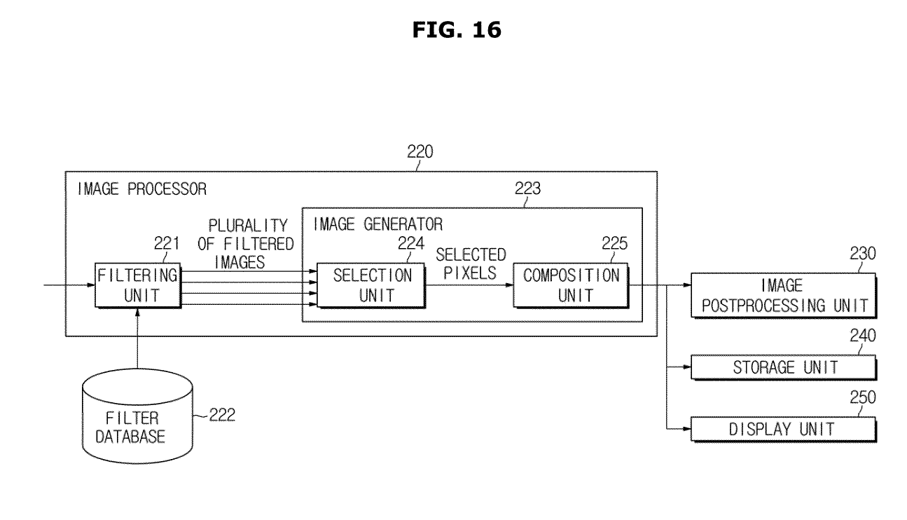

FIG. 16 is a block diagram illustrating a configuration of the image processor 220 of an ultrasonic imaging apparatus according to an exemplary embodiment.

In one embodiment, the image processor 220 may include a filtering unit 221 and an image generator 223. Also, the image processor 220 may include a processor, a microprocessor, a central processing unit (CPU), or an integrated circuit for executing programmable instructions.

The filtering unit 221 filters a beamformed ultrasonic signal, i.e., a beamformed ultrasonic image, using a predetermined filter to acquire a predetermined filtered signal, e.g., a filtered ultrasonic image. In one embodiment, as illustrated in FIG. 16, the filtering unit 221 may filter a beamformed ultrasonic signal using a plurality of filters, e.g., first and second filters to acquire a plurality of filtered ultrasonic signals corresponding in number to the number of filters. For example, the filtering unit 221 may acquire a first filtered ultrasonic signal by applying the first filter to the beamformed ultrasonic signal and acquire a second filtered ultrasonic signal using the second filter.

In one embodiment, as illustrated in FIG. 16, the filtering unit 221 may detect a predetermined filter from the filter database 222 and filter the beamformed ultrasonic signal using the detected predetermined filter. The filter database 222 may comprise various types of a plurality of filters as illustrated in FIG. 4 or point spread functions.

The filters used in the filtering unit 221 may be point spread functions, high resolution filters, LSFs, or cepstrum filters.

The plurality of filtered ultrasonic signals generated by the filtering unit 221 is transmitted to the image generator 223.

The image generator 223 may include a selection unit 224 and a composition unit 225.

The selection unit 224 may select pixel data for pixels of an image to be generated by the composition unit 225 from the filtered ultrasonic signals. The selection unit 224 may compare a plurality of filtered ultrasonic signals, i.e., filtered ultrasonic images, or at least one pixel of each filtered ultrasonic image and select at least one pixel from the filtered ultrasonic signals, i.e., the filtered ultrasonic images. The selected pixel may be used as a pixel of the image to be generated by the composition unit 225.

The selection unit 224 may compare data of corresponding pixels of each filtered ultrasonic image and select at least one pixel from at least one of the filtered ultrasonic images.

In addition, the selection unit 224 may select at least one pixel using minimum variance. For example, the selection unit 224 may select predetermined pixels for the entire area or a partial area of the image to be generated by the composition unit 225 by determining an optimal filtered ultrasonic image from the filtered ultrasonic images acquired by the filtering unit 221 using Equation 2 or 3 and selecting predetermined pixels from the determined optimal filtered ultrasonic image. In this regard, the optimal filtered ultrasonic image may be a filtered ultrasonic image having the smallest difference from the ideal ultrasonic image.

The composition unit 225 may generate an ultrasonic image by composing a plurality of pixels selected by the selection unit 224. The ultrasonic image generated by the composition unit 225 may be obtained through composition of the filtered ultrasonic images by different filters. In other words, the image generated by the composition unit 225 may be obtained through composition of pixels filtered by different filters. For example, a portion of the image generated by the composition unit 225 may be filtered by a predetermined filter, and another portion of the image may be filtered by a filter that differs from the predetermined filter. In some embodiments, all pixels of the image generated by the composition unit 225 may be selected from among pixels of an ultrasonic image filtered by a particular filter.

The ultrasonic image generated by the composition unit 225 may be an ultrasonic image having a variety of image modes. For example, the ultrasonic image may be an A-mode ultrasonic image or a B-mode ultrasonic image. The A-mode ultrasonic image refers to an ultrasonic image represented by amplitude. In particular, the A-mode ultrasonic image is an ultrasonic image in which a target site is represented by a distance between the ultrasonic probe P and the target site or the like while the intensity of reflection is represented in amplitude of the ultrasonic image. The B-mode ultrasonic image is an ultrasonic image represented in brightness. In particular, the B-mode ultrasonic image is an ultrasonic image in which the magnitude of ultrasonic echo waves is represented in brightness of the ultrasonic image. The B-mode ultrasonic image is commonly used because it allows the user to easily and visually identify inner tissues or structures of the target site within an object.

The ultrasonic image generated by the composition unit 225 may be transmitted to the image postprocessing unit 230, the storage unit 240, or the display unit dp.

The image postprocessing unit 230 may perform predetermined image processing on the ultrasonic image generated by the image processor 220. For example, the image postprocessing unit 230 may correct brightness, luminance, contrast, sharpness or the like of the entire area or a partial area of the ultrasonic image so that the user may distinctly view tissues in the ultrasonic image. The image postprocessing unit 230 may correct the ultrasonic image according to user instructions or commands or predefined settings. In addition, when a plurality of ultrasonic images is output from the image processor 220, the image postprocessing unit 230 may generate a three-dimensional stereoscopic ultrasonic image using the output ultrasonic images.

The storage unit 240 may temporarily or permanently store the ultrasonic image. The ultrasonic image stored in the storage unit 240 may be an ultrasonic image generated by the image processor 220 or an ultrasonic image corrected by the image postprocessing unit 230.

The display unit dp displays an ultrasonic image to the user. In some embodiments, the display unit dp may directly display the ultrasonic image generated by the image processor 220 to the user or may display an ultrasonic image upon which predetermined image processing has been performed by the image post-imaging unit 230 to the user. In addition, the display unit dp may display an ultrasonic image stored in the storage unit 240 to the user. The ultrasonic image displayed on the display unit dp may be the A-mode ultrasonic image, the B-mode ultrasonic image, or the three-dimensional stereoscopic ultrasonic image. In some embodiments, the display unit dp may be directly installed at the main body M as illustrated in FIG. 12. Alternatively, the display unit dp may be installed at a workstation connected to the main body M via a wired or wireless communication network.

The input unit i may receive predetermined user instructions or commands to control the ultrasonic imaging apparatus. For example, the input unit i may include various user interfaces such as various buttons, a keyboard, a mouse, a trackball, a touch screen, a paddle, and the like. In some embodiments, the input unit i may be directly installed at the main body M as illustrated in FIG. 12. Alternatively, the input unit i may be installed at a workstation connected to the main body M via a wired or wireless communication network.

As an example of the ultrasonic imaging apparatus, the ultrasonic imaging apparatus including the ultrasonic elements t10 installed at the ultrasonic probe P, and the beamforming unit 210, the image processor 220, the ultrasonic generation controller 110, the power source 111, and the image postprocessing unit 230 that are installed at the main body M has been described above. In another embodiment, an ultrasonic imaging apparatus including the beamforming unit 210, the image processor 220, the ultrasonic generation controller 110, and the power source 111 that are installed at the ultrasonic probe P may be used.

In addition, although a general ultrasonic imaging apparatus to which the above-described image processing unit is applied has been described above, the above-described image processing unit may also be applied to various kinds of ultrasonic imaging apparatuses such as an elastographic imaging apparatus and a photoacoustic imaging apparatus, in addition to the above-described general ultrasonic imaging apparatus. In addition, the above-described image processing unit may be applied directly or with partial modification to a radar, a sonar, or the like. In addition to the above-described apparatuses, the above-described image processing unit may be applied to various other apparatuses that correct an image using at least one filter.

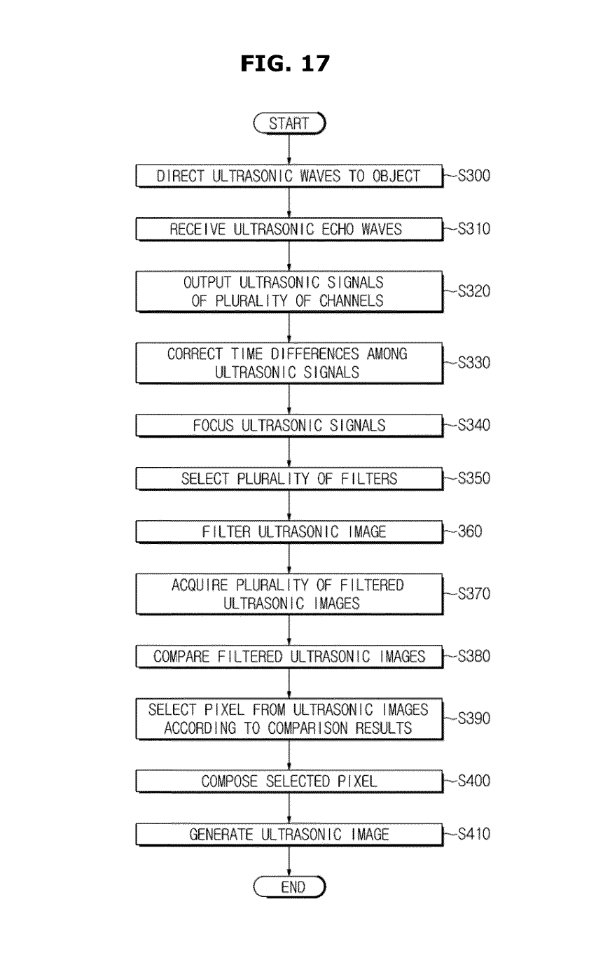

Hereinafter, an ultrasonic imaging apparatus control method according to an exemplary embodiment will be described with reference to FIG. 17. FIG. 17 is a flowchart illustrating a method of controlling an ultrasonic imaging apparatus according to an exemplary embodiment.

Referring to FIG. 17, at least one target site inside an object is irradiated with ultrasonic waves (operation S300), and ultrasonic echo waves reflected from the at least one target site irradiated with ultrasonic waves are received (operation S310). The ultrasonic waves may be emitted from and received by a predetermined ultrasonic element, e.g., an ultrasonic transducer. The ultrasonic element may perform both emission and reception of the ultrasonic waves, or alternatively, different ultrasonic elements may respectively emit and receive the ultrasonic waves.

The received ultrasonic echo waves are converted into an electric signal, e.g., an ultrasonic signal and then output (operation S320). When the ultrasonic echo waves are received by a plurality of ultrasonic elements, ultrasonic signals of a plurality of channels may be output from the ultrasonic elements.

Time differences among the output ultrasonic signals of the channels are corrected (operation S330), and the time difference-corrected ultrasonic signals are focused (operation S340). Consequently, a beamformed ultrasonic signal is output. The beamformed ultrasonic signal may be used as an ultrasonic image.

A plurality of filters is selected to filter the beamformed ultrasonic signal, i.e., an ultrasonic image (operation S350). In this case, filters appropriate for filtering the ultrasonic image may be selected from a filter database. In one embodiment, at least one of the filters may be a point spread function. In addition, at least one of the filters may be an LSF or a cepstrum filter.

The beamformed ultrasonic signal, i.e., an ultrasonic image, is filtered using the selected filters (operation S360).

Subsequently, the ultrasonic image may be filtered using each selected filter to acquire a plurality of filtered ultrasonic images (operation S370). In other words, the ultrasonic image may be filtered using the selected filters, e.g., first, second and third filters and acquire a plurality of filtered ultrasonic images corresponding in number to the number of the selected filters, e.g., first, second and third filtered ultrasonic images.

The filtered ultrasonic images are compared with one another (operation S380). The filtered ultrasonic images may comprise a plurality of pixels. In one embodiment, to compare the filtered ultrasonic images with one another, corresponding pixels of the plurality of pixels of the filtered ultrasonic images may be compared with one another. In addition, in one embodiment, variances of the corresponding pixels may be compared with one another.

According to comparison results of operation S380, predetermined pixels are selected and detected from the ultrasonic images (operation S390). In one embodiment, variances of the corresponding pixels thereof may be compared with one another and a pixel having the smallest variance among the variances of the corresponding pixels may be selected. In this regard, the variances may be determined by Equation 2 or 3 above. In addition, to select predetermined pixels, a filtered ultrasonic image to which an optimal filter has been applied may be determined from the filtered ultrasonic images and predetermined pixels may be selected according to ultrasonic image determination results. More particularly, a filtered ultrasonic image to which an optimal filter has been applied, among the filtered images, may be determined for the entire area or a partial area of an ultrasonic image to be acquired and pixels of the filtered ultrasonic image to which an optimal filter has been applied, corresponding to the entire area or partial area thereof, may be detected and selected.

The pixels selected in operation S390 are composed (operation S400). As a result, an ultrasonic image, a main lobe of which is emphasized and a side lobe of which is attenuated, may be generated (operation S410).

The image processing method and the imaging apparatus control method according to exemplary embodiments may be coded as software and be stored in a non-transitory readable medium. The non-transitory readable medium may be built in various types of image processing units or imaging apparatuses and support the image processing units or imaging apparatuses to carry out the methods as described above.

A non-transitory readable medium is a medium which does not store data temporarily such as a register, cash, and memory but stores data semi-permanently and is readable by devices. More specifically, the aforementioned various applications or programs may be stored and provided in a non-transitory readable medium such as a compact disk (CD), digital video disk (DVD), hard disk, Blu-ray disk, universal serial bus (USB), memory card, and read-only memory (ROM).

According to the above-described image processing unit, the ultrasonic imaging apparatus, and the image generation method according to exemplary embodiments, inaccuracy of a restored image, e.g., an ultrasonic image based on an ultrasonic signal may be improved through restoration of an image using filtering. In addition, an image that substantially approximates to an ideal image may be acquired.

In addition, by using the above-described image processing unit, the ultrasonic imaging apparatus, and the image generation method according to exemplary embodiments, a side lobe of a filtered image may be attenuated while maintaining high-frequency components of the filtered image.

Moreover, when an image is acquired using sound waves, a VHF, or the like, blurring of the image according to the velocity of sound waves, a VHF, or the like may be substantially prevented.

Furthermore, when the image processing unit according to exemplary embodiments is applied to an ultrasonic imaging apparatus, an ultrasonic image with higher resolution and higher quality may be easily acquired. Thus, an ultrasonic image with higher resolution that is the same as or substantially similar to an object to be imaged may be provided to a user, e.g., a doctor or the like who examines a patient using the ultrasonic imaging apparatus and accordingly, the user may diagnose a patient more accurately.

Although a few exemplary embodiments have been shown and described, it would be appreciated by those skilled in the art that many alternatives, modifications, and variations may be made without departing from the principles and spirit of the disclosure, the scope of which is defined in the claims and their equivalents.

What is claimed is:

1. An image processing unit comprising:
an input unit configured to receive image data of at least one image;
a plurality of filters configured to filter the image data to generate a plurality of filtered images; and
an image generator configured to compare the plurality of filtered images in a comparison to select at least one pixel from the plurality of filtered images according to results of the comparison.

2. The image processing unit according to claim 1, wherein the image generator comprises a selector configured to compare pixel data of a pixel of one of the plurality of filtered images with pixel data of corresponding pixels of remaining ones of the plurality of filtered images and select the at least one pixel from at least one of the plurality of filtered images.

3. The image processing unit according to claim 2, wherein the selector compares variances of pixel data of corresponding pixels of the plurality of filtered images and selects a pixel having a smallest variance among the variances of the pixel data from the at least one of the plurality of filtered images.

4. The image processing unit according to claim 2, wherein the selector compares pixel data of a plurality of pixels of the one of the plurality of filtered images with pixel data of a plurality of pixels of the remaining ones of the plurality of filtered images and selects the at least one pixel from the at least one of the plurality of filtered images.

5. The image processing unit according to claim 2, wherein the selector compares pixel data of adjacent pixels of the one of the plurality of filtered images with pixel data of corresponding adjacent pixels of the remaining ones of the plurality of filtered images and selects the at least one pixel from the at least one of the plurality of filtered images.

6. The image processing unit according to claim 2, wherein the image generator further comprises a composer configured to compose the pixel selected by the selector.

7. The image processing unit according to claim 1, further comprising a filter database to store the plurality of filters.

8. The image processing unit according to claim 1, wherein at least one of the plurality of filters is a point spread function (PSF).

9. The image processing unit according to claim 1, wherein at least one of the plurality of filters is a least square filter (LSF) or a cepstrum filter.

10. An ultrasonic imaging apparatus comprising:
a beamformer configured to beamform collected ultrasonic signals of a plurality of channels to output the beamformed ultrasonic signals; and
an image processor configured to generate a plurality of filtered ultrasonic images by filtering at least one of the beamformed ultrasonic signals using a plurality of filters, to compare the plurality of filtered ultrasonic images in a comparison to select at least one pixel from the plurality of filtered ultrasonic images according to results of the comparison.

11. The ultrasonic imaging apparatus according to claim 10, wherein the image processor comprises a selector configured to compare pixel data of a pixel of one of the plurality of filtered ultrasonic images with pixel data of corresponding pixels of remaining ones of the plurality of filtered ultrasonic images and select the at least one pixel from at least one of the plurality of filtered ultrasonic images.

12. The ultrasonic imaging apparatus according to claim 11, wherein the selector compares variances of pixel data of corresponding pixels of the plurality of filtered ultrasonic images and selects a pixel having a smallest variance among the variances of the pixel data from the at least one of the plurality of filtered ultrasonic images.

13. The ultrasonic imaging apparatus according to claim 11, wherein the image processor further comprises a composer configured to compose the pixel selected by the selector.

14. An image generation method comprising:
receiving image data of at least one image;
filtering the image data using a plurality of filters to generate a plurality of filtered images; and
selecting at least one pixel from the plurality of filtered images according to results of comparison between the plurality of filtered images.

15. The image generation method according to claim 14, wherein the selecting comprises comparing pixel data of a pixel of one of the plurality of filtered images with pixel data of corresponding pixels of remaining ones of the plurality of filtered images and selecting the at least one pixel from at least one of the plurality of filtered images.

16. The image generation method according to claim 15, wherein the selecting comprises comparing variances of pixel data of corresponding pixels of the plurality of filtered images and selecting a pixel having a smallest variance among the variances of the pixel data from the at least one of the plurality of filtered images.

17. The image generation method according to claim 15, wherein the selecting comprises comparing pixel data of a plurality of pixels of one of the plurality of filtered images with pixel data of a plurality of pixels of remaining ones of the plurality of filtered images and selecting the at least one pixel from the at least one of the plurality of filtered images.

18. The image generation method according to claim 17, wherein the selecting comprises comparing pixel data of adjacent pixels of one of the plurality of filtered images with pixel data of corresponding adjacent pixels of the remaining ones of the plurality of filtered images and selecting the at least one pixel from the at least one of the plurality of filtered images.

19. The image generation method according to claim 14, further comprising composing the selected pixel.

20. The image generation method according to claim 14, wherein at least one of the plurality of filters is a point spread function (PSF).

21. A non-transitory computer readable recording medium having recorded thereon a program executable by a computer for performing the image generation method according to claim 14.
